(19) United States
(12) Patent Application Publication
(10) Pub. No.: US 2010/0087752 A1
(43) Pub. Date: Apr. 8, 2010

(54) ASSESSMENT OF URINARY SYSTEM FUNCTION BY PATTERN MATCHING

(76) Inventors: Luya Li, Coquitlam (CA); Raymond Jacob Becker, Langley (CA); Matthew Peter Walls, New Westminster (CA)

Correspondence Address: OYEN, WIGGS, GREEN & MUTALA LLP;480 - THE STATION, 601 WEST CORDOVA STREET, VANCOUVER, BC, V6B 1G1, CA

(21) Appl. No.: 12/532,624

(22) PCT Filed: Mar. 20, 2008
(86) PCT No.: PCT/CA08/00533

§ 371 Date: Oct. 9, 2009

Related U.S. Application Data
(60) Provisional application No. 60/896,451, filed on Mar. 22, 2007.
(60) Provisional application No. 60/939,041, filed on May 18, 2007.

(51) Int. Cl. A61B 5/05; A61B 5/103

(52) U.S. Cl. 600/561; 600/587

(57) ABSTRACT

A system for monitoring urinary system operation comprises a sensor for detecting a level of one or more compounds in tissues of a subject's bladder. A processor identifies a pattern in a time variation of the level detected by the sensor. The processor generates an indicator of bladder function based at least in part on the identified pattern. In some embodiments the processor generates a possible diagnosis based upon the identified pattern and one or more pieces of additional information. The additional information may comprise one or more of: a residual amount of urine after urination; a maximum uroflow rate; and, a delay in onset of urination.

### CROSS-REFERENCE TO RELATED APPLICATIONS

This application claims priority from U.S. patent application No. 60/896,451 filed 22 Mar. 2007 and entitled ASSESSMENT OF URINARY SYSTEM FUNCTION BY PATTERN MATCHING and U.S. patent application No. 60/939,041 filed 18 May 2007 and entitled ASSESSMENT OF URINARY SYSTEM FUNCTION BY PATTERN MATCHING. For purposes of the United States of America, this application claims the benefit under 35 U.S.C. §119 of U.S. patent application No. 60/896,451 filed 22 Mar. 2007 and entitled ASSESSMENT OF URINARY SYSTEM FUNCTION BY PATTERN MATCHING and U.S. patent application No. 60/939,041 filed 18 May 2007 and entitled ASSESSMENT OF URINARY SYSTEM FUNCTION BY PATTERN MATCHING both of which are hereby incorporated herein by reference.

### TECHNICAL FIELD

The invention relates to assessing urinary system function. Embodiments of the invention have application in the diagnosis of obstruction of the lower urinary tract. The invention may be embodied in diagnostic apparatus and methods.

### BACKGROUND

Obstruction of the urinary tract (or of the urinary system generally) may cause a variety of problems, including urinary tract infections, urinary retention, and urine reflux. Urinary tract obstruction may also be a symptom of prostate cancer in some cases.

An obstruction anywhere along the urinary tract from the kidneys to the urethra, can result in increase pressure within the urinary tract and may slow the flow of urine. Obstructions may completely or partially block the urinary tract. Damage to the kidneys, stone formation and urinary tract infections may result from urinary tract obstructions. Obstructions in the lower urinary tract may be caused by, for example, benign prostatic hypertrophy (BPH), Prostate cancer and other pelvic malignancies, congenital urtheral valve abnormalities etc. Obstruction of the urinary tract may also cause a variety of problems during the voiding cycle, including urinary tract infections, urinary retention, and urine reflux.

There exists a need for systems and methods for assessing performance of the urinary system. There exists a need for systems and methods for evaluating obstruction of the urinary tract.

### BRIEF DESCRIPTION OF DRAWINGS

The accompanying drawings illustrate non-limiting embodiments of the invention.

FIG. 1 schematically depicts the components of a system according to one embodiment of the invention.

FIGS. 2A through 2I are graphs illustrating typical patterns that can be observed in signals from the system of FIG. 1.

FIG. 3 schematically depicts the components of a system according to another embodiment of the invention.

FIGS. 4A and 4B are graphs showing variations in levels of Hb, HbO₂, HbSum, and Cyt as a function of time during voiding (urination) for different subjects.

FIGS. 5A, 5B, 5C and 5D are examples of reports that may be output by a system according to an embodiment of the invention.

FIG. 6 is a flowchart illustrating a method for establishing a preliminary diagnosis according to an embodiment of the invention.

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

This invention provides systems and methods for providing information useful for diagnosing function of the urinary system. In certain embodiments of this invention, which are described below, the systems and methods are applied to diagnosing function of the lower urinary tract, and the systems and methods generate indications of whether there is obstruction of the lower urinary tract based upon patterns in levels of one or more compounds in the tissues of a subject's bladder. Results of the assessment may be stored for archival purposes and future reference and/or displayed or otherwise output to a user such as the subject's physician. The systems and methods described below may also be applied to diagnosing function of other parts of the urinary system (or the urinary system generally) based upon patterns in levels of one or more compounds in the bladder tissues or other tissues in or around the urinary system.

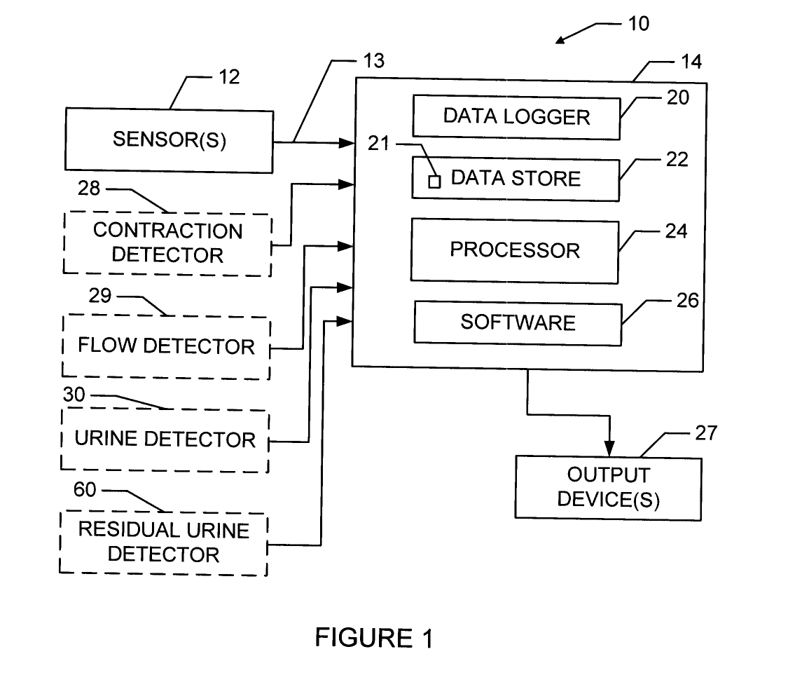

FIG. 1 shows a system 10 according to one embodiment of the invention. System 10 comprises a sensor 12 that detects levels of one or more compounds in the tissues of a subject's bladder. Sensor 12 provides signals 13 that indicate detected levels of the one or more compounds. The compounds have levels that are related to blood circulation and/or muscle activity in the bladder. The compounds may comprise, for example:

oxygenated haemoglobin (HbO₂);

deoxygenated haemoglobin (Hb);

total haemoglobin (HbSum);

cytochrome (Cyt);

myoglobin (Mb);

other chromophores;

and the like.

Sensor 12 may comprise an optical sensor or may sense levels of the one or more compounds in any other suitable manner (for example, HbSum levels can be determined by ultrasound techniques). Sensor 12 may comprise a single sensing device or a plurality of sensing devices that sense levels of different compounds. Sensor 12 may be located in any suitable location such as a trans-urethral or trans-vaginal sensor, a sensor in the bladder, a trans-rectal sensor (located in the anus), or the like. Sensor 12 may comprise one or multiple sensors that measure levels of the compound(s) at one or multiple locations in the subject's bladder. In some embodiments, sensor 12 comprises a near infrared spectroscopy (NIRS) sensor and a NIRS system that determines levels of the one or more compounds in the subject's bladder and passes signals indicative of those levels to a data logger and/or an analysis unit. In some embodiments the NIRS system is integrated with a data logger or data analysis unit.

System 10 includes an analysis unit 14 that receives signals 13 from sensor 12 and determines an indicator useful for assessment of lower urinary tract function based upon the signals. In the illustrated embodiment, analysis unit 14 includes a data logger 20 comprising a data store 22, a processor 24, analysis software 26 executed by processor 24 and an output device 27.

In operation, data logger 20 receives signals 13 and logs values 21 (which may be stored in data store 22) indicative of the concentrations of the one or more monitored compounds. Logged values 21 can be analyzed to determine how the concentrations of the monitored compounds change over time. Processor 24 analyzes patterns in the logged values. In some embodiments, the patterns are monitored over an interval during which the subject voids. The inventors have determined that there is a correlation between urinary obstruction and the patterns according to which compounds indicative of blood flow and/or muscle activity change in the tissues of the subject's bladder during voiding.

Data analysis unit 14 may determine an interval over which to analyze logged data 21. In some embodiments, data analysis unit 14 detects a first time corresponding to an onset of voiding and a second time corresponding to an end of voiding and analyzes logged data 21 between the first and second times.

The first time may be identified in a range of ways including one or more of:
receiving a manual input (e.g. on a switch keypad or the like) indicating that the subject has been given permission to void, voiding is about to start or has just started;
detecting a signature in signal 13 that is characteristic of the onset of voiding;
receiving a signal from a bladder contraction detector 28. Contraction detector 28 (which is indicated in dotted lines to show that it is optional) may be configured to detect bladder contractions by directing energy towards the subject's bladder and detecting transmitted, scattered and/or reflected energy returning from the subject's bladder. For example, contraction detector 28 may be implemented using:
   an optical energy source and an optical detector such as an infrared or near infrared detector; or
   an ultrasonic energy source and an ultrasonic detector such as an ultrasound transducer.
In some embodiments, contraction detector 28 may be in physical contact with the subject, and comprise a mechanical detector to which movements of the subject's bladder are physically coupled.
detecting uroflow by means of a suitable flow detector 29 (which is indicated in dotted lines to show that it is optional). Flow detector 29 may be optical or ultrasonic or electro-mechanical, for example.
detecting the presence of urine by way of a suitable urine detector 30 (which is indicated in dotted lines to show that it is optional). Detector 30 may, for example: detect urine by a change in electrical conductivity between two or more electrodes in a path of the urine; detect urine optically or ultrasonically.
detecting a signature in an EMG signal or the like that is characteristic of the onset of voiding.

The second time may be identified in a range of ways including:
receiving a manual input (e.g. on a switch keypad or the like) indicating that voiding is finishing.
setting the second time at a predetermined interval after the first time, the interval being long enough to observe the desired pattern.
detecting uroflow by means of flow detector 29 and monitoring an output of flow detector 29 to determine when uroflow has substantially ceased or has fallen to below some threshold value.
detecting the presence of urine by way of urine detector 30 and determining when urine is no longer present at detector 30.
detecting a signature in signal 13 that is characteristic of the end of voiding.

Data analysis unit 14 may have various outputs. For example, data analysis unit 14 may display graphs showing the variation in time of the concentrations of one or more monitored compounds on a suitable display. In some embodiments, data analysis unit 14 has a user interface which permits a user such as a physician or technician to identify significant points on the concentration curves for one or more monitored compounds. The user may, for example, use controls of the user interface to position a cursor or cursors to indicate locations of interest on the concentration curve(s). Data analysis unit 14 may then analyze the locations of the selected points to identify a pattern of variation in the concentration of a monitored compound. Data analysis unit 14 may automatically identify a pattern matched by the variation in the concentration of a monitored compound.

In some embodiments, data analysis unit 14 generates an indication of the degree of or the likelihood of urinary obstruction. The indicator may be based in whole or part on pattern(s) matched by the variations in concentration of one or more monitored patterns during voiding (i.e. between the time the subject is given permission to void and the completion of urination).

The inventors have determined that, during voiding, the concentrations of HbO₂ and other chromophores in bladder tissues vary in patterns that are indicative of whether or not the subject suffers from lower urinary obstruction. For example, in unobstructed subjects, concentrations of HbO₂ tend to trend upward upon the commencement of voiding. In subjects who have some degree of lower urinary obstruction the concentrations of HbO₂ tend to trend downward upon the commencement of voiding. Patterns of concentrations of HbO₂ and other chromophores in bladder tissues or other regions of the urinary system may also be indicative of obstructions in other parts of the urinary system.

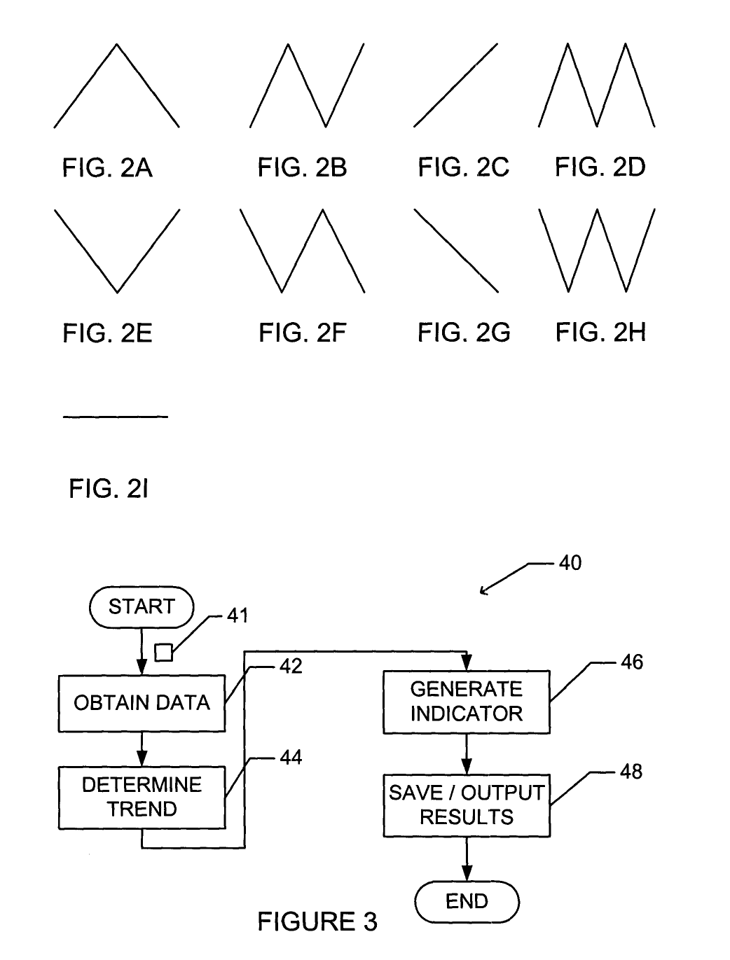

FIGS. 2A through 2I show example trends in concentrations of HbO₂ and other chromophores that may be observed during voiding. The patterns of FIGS. 2A through 2D begin with an increasing (upward) trend whereas the patterns of FIGS. 2E through 2H begin with a decreasing (downward) trend.

Table I shows the patterns of changes in levels of HbO₂, Hb, HbSum and Cyt observed during voiding for 13 men who had been separately diagnosed as having lower urinary obstruction. Each cell in Table I indicates the percentage of the subjects for whom the corresponding pattern was observed in the level of the corresponding compound. It can be seen that the patterns of FIGS. 2E to 2H (initial downward trend) predominate for HbO₂, Hb, and HbSum. It can also be seen that the patterns of FIGS. 2A to 2D (initial upward trend) predominate for Cyt.

TABLE I
Subjects Having Obstruction

| Pattern | Hb | HbO₂ | Cyt | HbSum |
|---|---|---|---|---|
| FIG. 2A | 15.4% | 0.0% | 69.2% | 0.0% |
| FIG. 2E | 69.2% | 61.5% | 7.7% | 53.8% |
| FIG. 2C | 0.0% | 0.0% | 0.0% | 0.0% |
| FIG. 2G | 0.0% | 7.7% | 0.0% | 0.0% |
| FIG. 2I | 0.0% | 7.7% | 0.0% | 0.0% |
| FIG. 2B | 0.0% | 0.0% | 0.0% | 0.0% |
| FIG. 2F | 0.0% | 0.0% | 0.0% | 15.4% |
| FIG. 2D | 0.0% | 7.7% | 7.7% | 15.4% |
| FIG. 2H | 15.4% | 15.4% | 15.4% | 7.7% |
| Down | 84.6% | 84.6% | 23.1% | 76.9% |
| Up | 15.4% | 7.7% | 76.9% | 15.4% |

Table II shows the patterns of changes in levels of HbO₂, Hb, HbSum and Cyt observed during voiding for 4 men who had been separately diagnosed as not having lower urinary obstruction (i.e. no obstruction). Each cell in Table II indicates the percentage of the subjects for whom the corresponding pattern was observed in the level of the corresponding compound. It can be seen that the patterns of FIGS. 2A to 2D (initial upward trend) predominate for HbO₂, Hb, and HbSum. It can also be seen that the patterns of FIGS. 2E to 2H (initial downward trend) predominate for Cyt.

TABLE II
Subjects Without Obstruction

| Pattern | Hb | HbO2 | Cyt | HbSum |
|---|---|---|---|---|
| FIG. 2A | 0.0% | 25.0% | 0.0% | 25.0% |
| FIG. 2E | 25.0% | 0.0% | 25.0% | 0.0% |
| FIG. 2C | 0.0% | 0.0% | 0.0% | 0.0% |
| FIG. 2G | 0.0% | 0.0% | 0.0% | 0.0% |
| FIG. 2I | 0.0% | 0.0% | 0.0% | 0.0% |
| FIG. 2B | 50.0% | 50.0% | 0.0% | 50.0% |
| FIG. 2F | 0.0% | 0.0% | 50.0% | 0.0% |
| FIG. 2D | 25.0% | 25.0% | 0.0% | 25.0% |
| FIG. 2H | 0.0% | 0.0% | 25.0% | 0.0% |
| Down | 25.0% | 0.0% | 100.0% | 0.0% |
| Up | 75.0% | 100.0% | 0.0% | 100.0% |

Table III shows the patterns of changes in levels of HbO₂, Hb, HbSum and Cyt observed during voiding for 9 men who had been separately diagnosed as having normal lower urinary tract function (i.e. healthy normal). Each cell in Table III indicates the percentage of the subjects for whom the corresponding pattern was observed in the level of the corresponding compound. It can be seen that the patterns of FIGS. 2A to 2D (initial upward trend) predominate for HbO₂, Hb, and HbSum. It can also be seen that the patterns of FIGS. 2E to 2H (initial downward trend) predominate for Cyt.

TABLE III
Normal Subjects

| Pattern | Hb | HbO2 | Cyt | HbSum |
|---|---|---|---|---|
| FIG. 2A | 0.0% | 0.0% | 0.0% | 0.0% |
| FIG. 2E | 11.1% | 0.0% | 0.0% | 0.0% |
| FIG. 2C | 11.1% | 0.0% | 0.0% | 0.0% |
| FIG. 2G | 0.0% | 0.0% | 0.0% | 0.0% |
| FIG. 2I | 0.0% | 0.0% | 0.0% | 0.0% |
| FIG. 2B | 11.1% | 11.1% | 0.0% | 22.2% |
| FIG. 2F | 0.0% | 0.0% | 11.1% | 0.0% |
| FIG. 2D | 66.7% | 88.9% | 0.0% | 77.8% |
| FIG. 2H | 0.0% | 0.0% | 88.9% | 0.0% |
| Down | 11.1% | 0.0% | 100.0% | 0.0% |
| Up | 88.9% | 100.0% | 0.0% | 100.0% |

It can be seen from Tables I to III that the patterns of change in chromophores such as HbO₂, Hb, HbSum and Cyt can be used to distinguish between subjects having lower urinary obstruction and subjects without lower urinary obstruction. This makes possible methods for assessing whether a subject suffers from lower urinary obstruction (and for assessing the function of the urinary system more generally) which involves identifying patterns of change in chromophores such as Hb, HbO₂, HbSum and Cyt during voiding. These methods may also be applied in assessing the function of other parts of the urinary system or the urinary system generally, by identifying patterns of changes in chromophores in the bladder or other tissues of the urinary system.

FIG. 3 shows an example method 40 according to an embodiment of the invention. In block 42, method 40 obtains data 41 indicative of levels of one or more compounds in the tissues of the bladder of an individual over a period during which the individual voids. In block 44 method 40 determines a trend in data 41 during the period immediately after the onset of voiding. Block 46 generates an indicator of lower urinary function based upon the trend determined in block 44. The indicator may comprise any information that could assist a physician to understand the function of the urinary system in the subject. For example, the indicator may comprise one or more of a numeric value, a qualitative indicator, raw data (as shown for example in Tables I, II and III), a graphic indicating the observed pattern, or the like. In block 48 the indicator is displayed or stored or both displayed and stored.

In some embodiments block 44 comprises fitting a function comprising one or more connected line segments to a curve indicating a variation on one of the levels with time. In some embodiments the fitted curve comprises a plurality of connected line segments such as two, three or four connected line segments. In some embodiments the analysis unit is configured to fit one of a plurality of fitting functions to the curve, the plurality of fitting functions can each comprising a different number of line segments. For example, the analysis unit may provide a plurality of fitting functions that includes a first fitting function comprising one line segment, a second fitting function comprising two connected line segments, and a third fitting function comprising three connected line segments. The analysis unit may determine which of the plurality of fitting functions provide the best fit to the curve according to a suitable measure of goodness of fit.

In some embodiments the analysis unit is configured to determine whether a slope of each line segment of the fitted function is positive or negative and to identify a fitted pattern in the slopes of the segments. For example, the fitted pattern may comprise one of the following:
one line segment, negative slope;
one line segment, positive slope;
two line segments, positive slope followed by negative slope;
two line segments, positive slope followed by negative slope;
three line segments, positive slope followed by negative slope followed by positive slope;
three line segments, negative slope followed by positive slope followed by negative slope;
four line segments, positive slope followed by negative slope followed by positive slope followed by negative slope; and,
four line segments, negative slope followed by positive slope followed by negative slope followed by positive slope.

In some embodiments, the indicator is based upon patterns observed for two or more compounds. For example, in some embodiments, the indicator is based upon patterns in levels of HbO₂ and Hb.

In some embodiments, the indicator is based upon patterns observed for two or more incidents of voiding. To facilitate such embodiments, system 10 may maintain raw data (or patterns matched to the raw data) for one or more previous incidents of voiding.

System 10 may be configured to perform a method such as method 40. For example, software 26 may cause processor 24 to implement a method according to the invention. The method may involve matching data received from sensor 12 to one of a plurality of patterns and generating an indicator indicative of the functioning of the lower urinary system based at least in part on which of the plurality of patterns are matched by the data received from sensor 12.

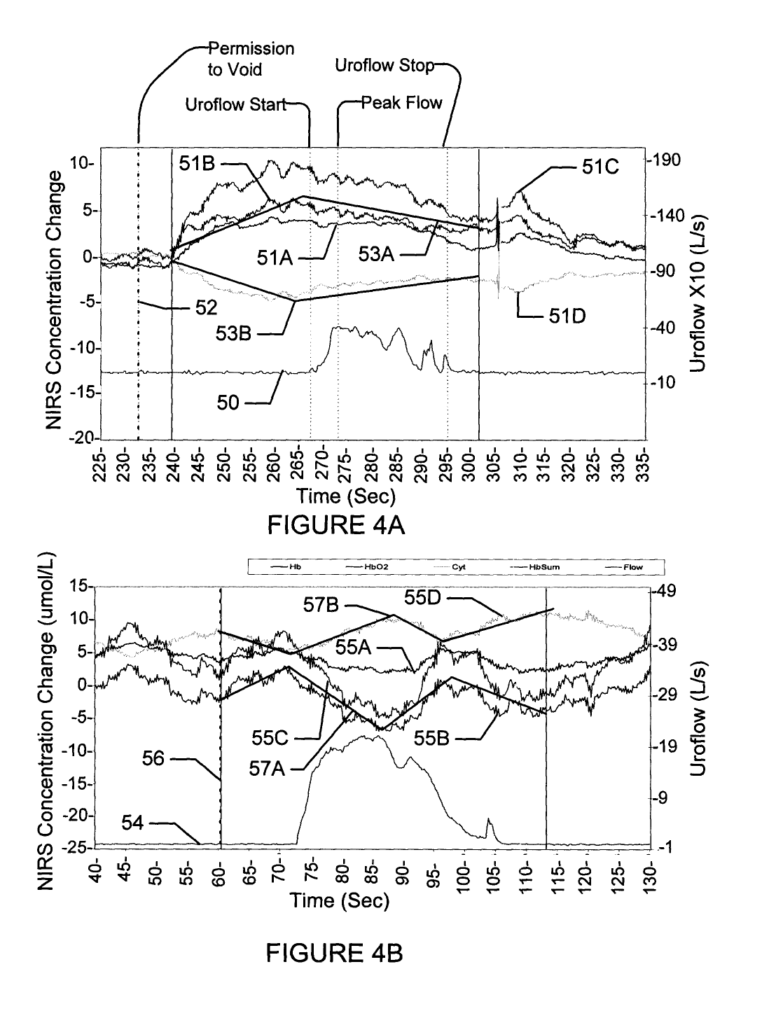

FIGS. 4A and 4B show example data. In FIG. 4A, curve 50 indicates urine flow while curves 51A, 51B, 51C and 51D respectively represent measured levels of Hb, HbO₂, HbSum and Cyt. The time that the subject is given “permission to void” is indicated by the vertical line 52. At the time corresponding to line 52 the subject may be invited to void. It can be seen that the portion of curves 51A, 51B, and 51C immediately after line 52 have the pattern of FIG. 2A (as indicated by the fit line 53A) whereas the corresponding portion of curve 51D has the pattern of FIG. 2E (as indicated by the fit line 53B).

In some embodiments system 10 has a user interface control which permits a user to indicate the time at which permission to void is given to a subject. In such embodiments, system 10 may record the time at which the control is invoked. In some embodiments, system 10 has a signal, such as a lamp, an auditory signal, a combination thereof or the like that is triggered by system 10 to indicate permission to void. In such embodiments, the subject may be told that they have permission to void as soon as the signal is delivered. System 10 may record the time that the signal is generated. In such embodiments, system 10 may display a line or other indicia on a display having a time axis to indicate when the subject was given permission to void.

In FIG. 4B, curve 54 indicates urine flow while curves 55A, 55B, 55C and 55D respectively represent measured levels of Hb, HbO₂, HbSum and Cyt. The time at which the subject is given permission to void is indicated by the vertical line 56. It can be seen that the portion of curves 55A, 55B, and 55C immediately after line 56 have the pattern of FIG. 2D (as indicated by fit line 57A) whereas the corresponding portion of curve 55D has the pattern of FIG. 2H (as indicated by fit line 57B).

System 10 may establish a possible diagnosis on the basis of a matched pattern plus additional information. The additional information may, for example, comprise one or more of:
a maximum rate of flow of urine during voiding;
a residual amount of urine remaining in the subject's bladder after voiding; and
a delay between initial contractions of the bladder and the onset of uroflow.
   Such additional information may be acquired directly by system 10 (using suitable sensors interfaced to system 10) or may be acquired by separate means and entered into system 10 by way of a user interface or data communication channel.

In the embodiment illustrated in FIG. 1, system 10 includes an optional sensor 60 that detects urine in the bladder. Sensor 60 may comprise, for example an ultrasonic sensor that detects sound signals reflected from the bladder that are indicative of an amount of urine present in the bladder. Data analysis unit 14 may be configured to detect the end of voiding and to operate sensor 60 to evaluate whether or not a significant amount of residual urine remains in the bladder after the end of voiding. The end of voiding may be determined in any of the ways described above in relation to determining the second time, for example.

A significant quantity of residual urine after the completion of voiding is a symptom of urinary obstruction. Data analysis unit 14 may be configured to generate an indication of the degree of or the likelihood of urinary obstruction based in whole or part on one or more of:
whether or not there is residual urine in the bladder after the end of voiding;
an amount of residual urine present in the bladder after the end of voiding;
pattern(s) matched by the variations in concentration of one or more monitored patterns during voiding;
or the like.

In the illustrated embodiment, system 10 has an optional uroflow sensor 29 that monitors a rate of uroflow during voiding. System 10 may determine one or both of the average flow rate or the maximum flow rate of urine during voiding by monitoring a signal output by sensor 29.

System 10 may optionally monitor the delay between the onset of bladder contractions and the start of urination in any suitable manner. For example, one means for determining such a delay is described in co-pending U.S. patent application No. 60/920,777 filed on 30 Mar. 2007 which is hereby incorporated herein by reference.

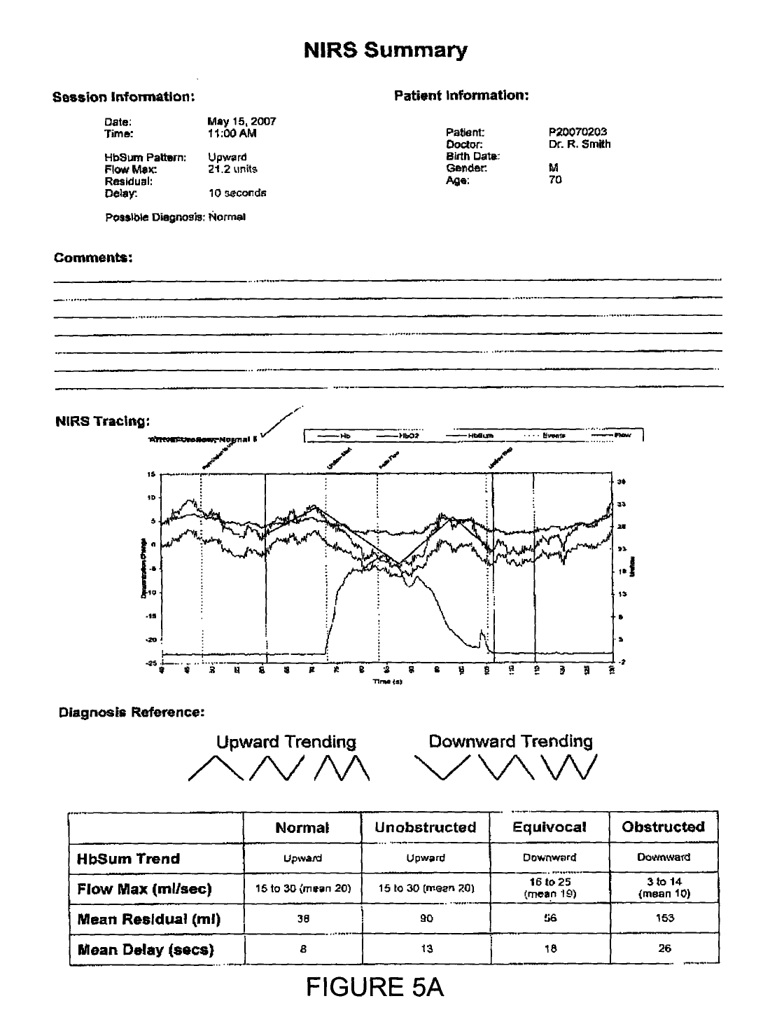

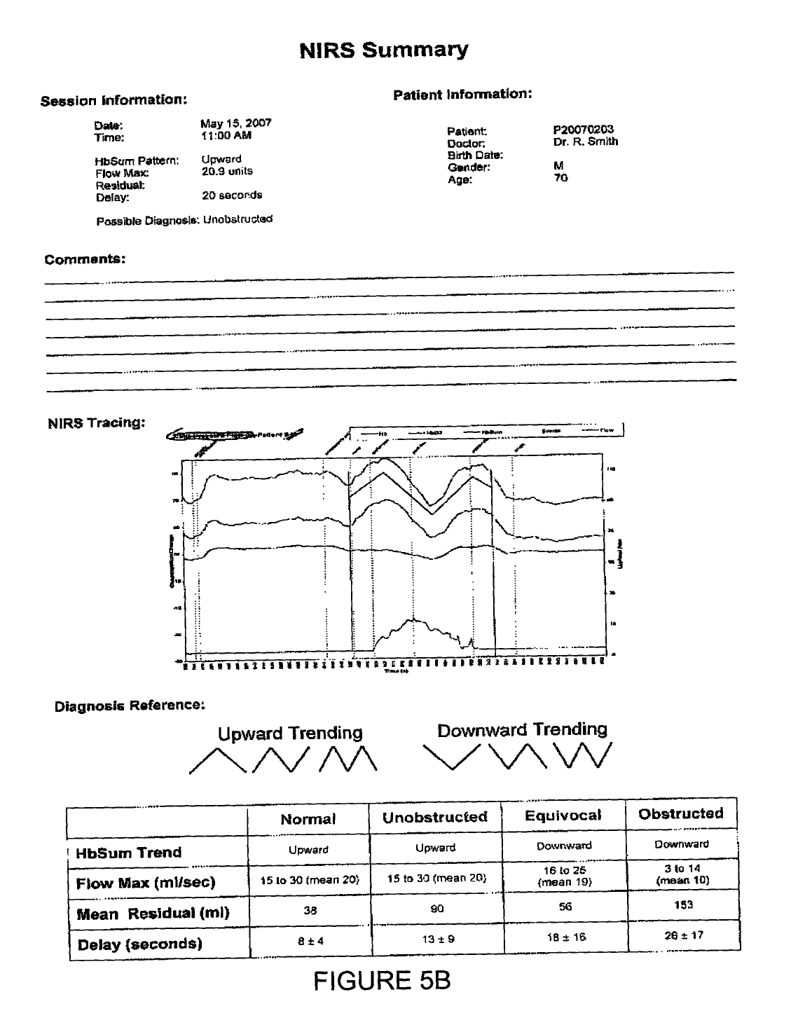

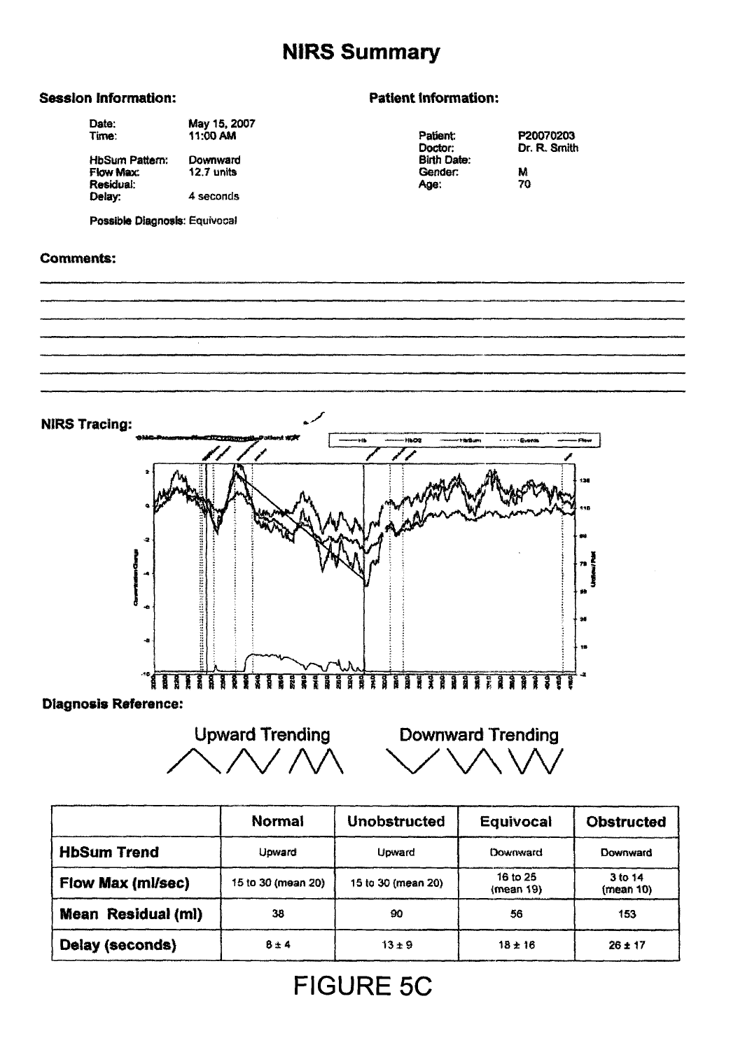

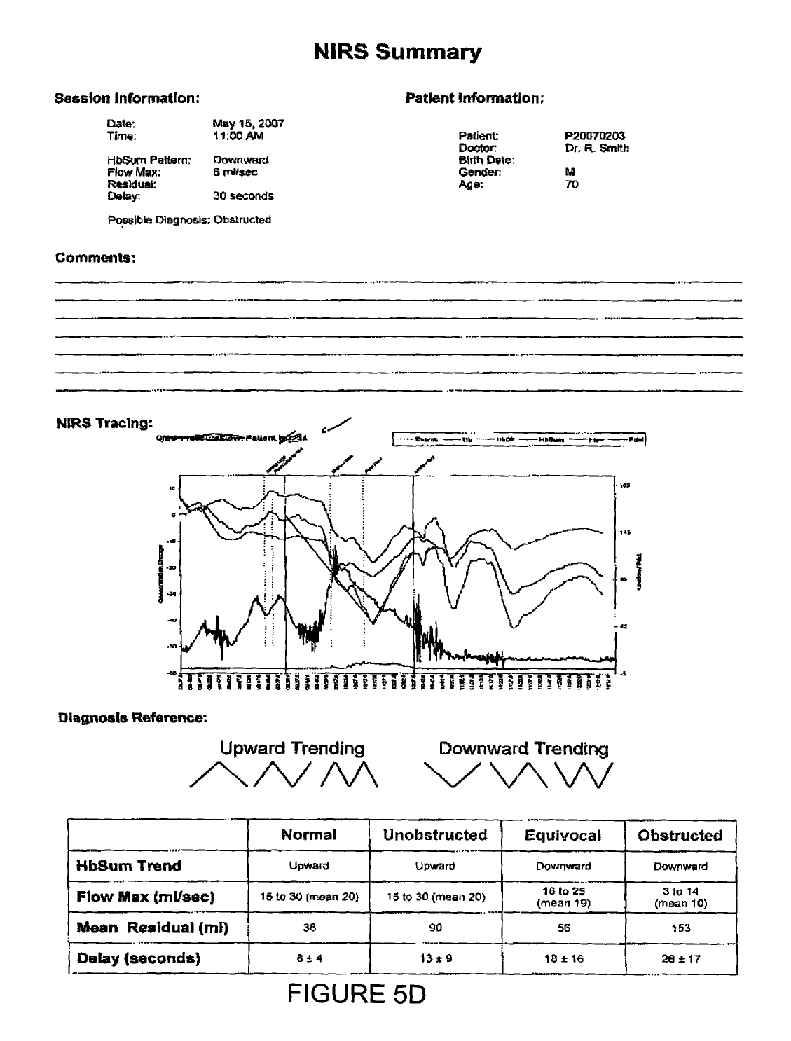

There is a range that extends between a subject suffering from obstruction, on one hand, and a subject having normal urinary tract function, on the other hand. In some embodiments, system 10 uses pattern matching, as described above, to determine whether the subject exhibits an “upward” or “downward” trending pattern, as shown in FIGS. 5A to 5C. The trend of the pattern (upward or downward) is used to classify the subject as belonging to a portion of this range toward a normal end of the range or a portion of the range toward an obstructed end of the range.

In the case of an upward-trending pattern, system 10 classifies the subject in a normal to unobstructed end of the range. In the case of a downward-trending pattern, system 10 classifies the subject in an equivocal to obstructed end of the range. System 10 may then use one or more pieces or additional information, such as one or more of maximum uroflow, delay in onset of uroflow and residual urine amount, to more specifically classify the subject within the portion of the range identified by pattern matching.

Higher maximum flow indicates a classification toward the normal end of the portion of the range. Lower maximum flow indicates a classification toward the obstructed end of the portion of the range. Maximum urine flow is particularly useful for distinguishing between degrees of obstruction in the portion of the range toward the obstructed end of the range.

Higher residual urine indicates a classification toward the normal end of the portion of the range. Lower residual urine indicates a classification toward the obstructed end of the portion of the range.

Shorter onset delay indicates a classification toward the normal end of the portion of the range. Longer onset delay indicates a classification toward the obstructed end of the portion of the range.

System 10 may comprise a logic system which determines a possible diagnosis by applying logical conditions to the nature of a pattern determined as described above and one or more additional information inputs. The logic system may comprise software which causes a processor in data analysis unit 14 to perform logical operations or hardware logic circuits or some combination thereof. In performing the classification, the logic system may compare additional information (such as maximum uroflow, uroflow onset delay, and/or residual urine) to corresponding thresholds or may base a classification of a subject in part on a value computed from one or more items of additional information.

System 10 may provide printed or displayed reports. The reports may include a possible diagnosis as determined by a logic system. FIGS. 5A to 5D show possible example reports for cases where a possible diagnosis ranges from normal (FIG. 5A) to unobstructed (FIG. 5B) to equivocal (FIG. 5C) to obstructed (FIG. 5D). In these examples, there are four possible diagnoses and system 10 classifies a subject as belonging to one of these four diagnoses. In the illustrated embodiment, each of a plurality of possible diagnoses have a narrative descriptive title (“normal”, “unobstructed”, “equivocal” and “obstructed”). In alternative embodiments there are a different number of possible diagnoses or the possible diagnosis is indicated by a value on an essentially continuous scale (e.g. a scale having “normal” represented by a value at one end of the scale, “obstructed” represented by a value at another end of the scale, and conditions between “normal” and “obstructed” expressed by intermediate values).

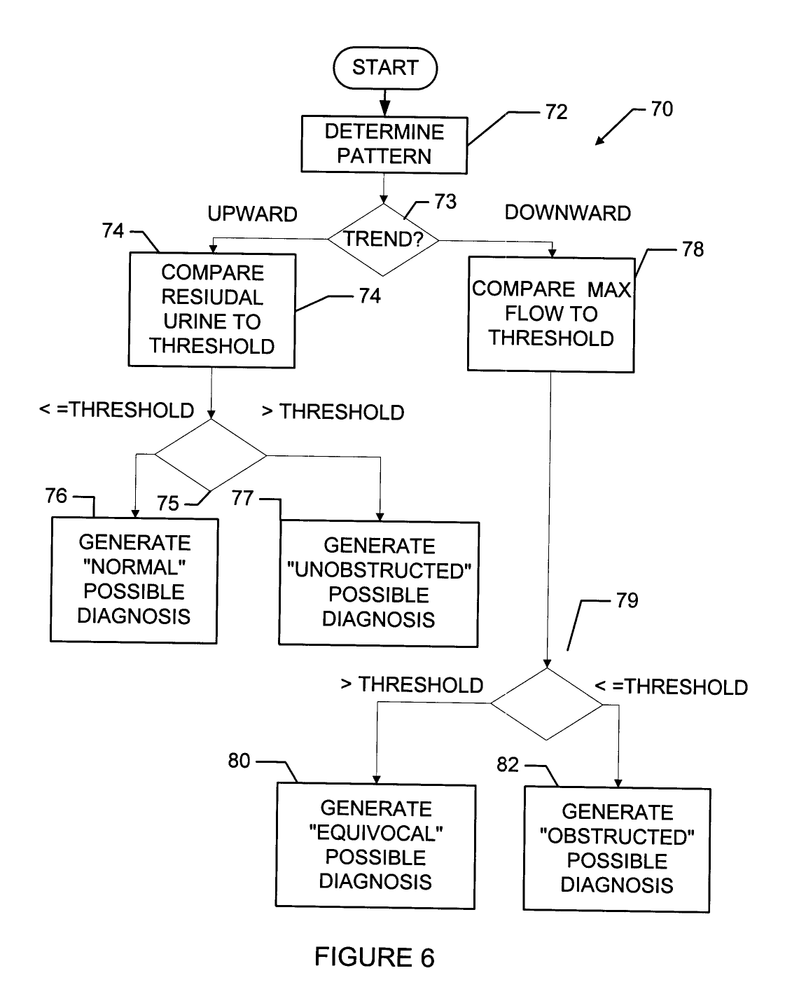

FIG. 6 shows an example method 70 according to a simple embodiment of the invention. At block 72 a pattern in a concentration of one or more biocompounds in a subject is identified. This may be done as described above. If the pattern identified in block 72 has an “upward” trend then method 70 branches at block 73 to block 74. Block 74 compares a measured amount of residual urine to a threshold. If the amount of residual urine is less than or equal to a threshold then block 75 branches to block 76 which generates a “normal” possible diagnosis.

In the event that the amount of residual urine exceeds the threshold then an “unobstructed” possible diagnosis is generated at block 77.

Where block 75 determines that the trend of the pattern identified in block 73 is “downward” then method 70 branches to block 78 which compares the maximum uroflow to a threshold. If the maximum uroflow exceeds the threshold then method 70 branches to block 80 which generates an “equivocal” possible diagnosis. Otherwise, method 70 branches to block 82 which generates an “obstructed” possible diagnosis. At any stage where an amount is compared to a threshold in method 70 the case that the amount equals the threshold to which it is being compared may be associated with either branch.

The possible diagnosis generated by method 70 may be printed on a report, displayed on a display, stored in a memory, or otherwise made available for use by a person or other process.

In other methods according to the invention, values for onset delay may be used to refine or confirm the possible diagnosis. In some methods according to the invention, data for multiple voiding cycles is accumulated for a subject and the possible diagnosis is based on the accumulated data.

Certain implementations of the invention comprise computer processors which execute software instructions which cause the processors to perform a method of the invention. For example, one or more processors in an analysis unit 14 may implement the methods of FIG. 3 or 6 by executing software instructions in a program memory accessible to the processors. The invention may also be provided in the form of a program product. The program product may comprise any medium which carries a set of computer-readable signals comprising instructions which, when executed by a data processor, cause the data processor to execute a method of the invention. Program products according to the invention may be in any of a wide variety of forms. The program product may comprise, for example, physical media such as magnetic data storage media including floppy diskettes, hard disk drives, optical data storage media including CD ROMs, DVDs, electronic data storage media including ROMs, flash RAM, or the like. The computer-readable signals on the program product may optionally be compressed or encrypted.

Where a component (e.g. a software module, processor, assembly, device, circuit, etc.) is referred to above, unless otherwise indicated, reference to that component (including a reference to a “means”) should be interpreted as including as equivalents of that component any component which performs the function of the described component (i.e., that is functionally equivalent), including components which are not structurally equivalent to the disclosed structure which performs the function in the illustrated exemplary embodiments of the invention.

While a number of exemplary aspects and embodiments have been discussed above, those of skill in the art will recognize that the invention has a wide range of aspects and includes, without limitation, aspects such as those set out above as well as combinations and sub-combinations of the features of those aspects, as well as certain modifications, permutations, and additions to the example embodiments described herein. For example:
Instead of logging data 21, analysis unit 14 may analyze information in signals 13 as the signals are received.
Analysis unit 14 is not necessarily connected directly to sensor 12 but may receive data that indicates levels of suitable compounds that has been previously acquired by a suitable sensor 12, which may comprise a data logger.
   It is therefore intended that the following claims be interpreted to include all such modifications, permutations, additions and sub-combinations.

1. Apparatus for monitoring urinary tract function, the apparatus comprising:
an input for receiving sensor signals indicative of levels of one or more compounds in tissues of a subject's bladder; and
an analysis unit configured to:
receive a signal indicating time variations in the levels indicated by the sensor signals;
identify a pattern in the time variations; and
generate an indicator of urinary tract function based at least in part on the identified pattern.

2. Apparatus according to claim 1, wherein the analysis unit is configured to identify time trends in the levels indicated by the sensor signals and to determine whether the time trends are increasing or decreasing during a period after an onset of voiding.

3. Apparatus according to claim 2, wherein the one or more compounds for which the sensor signals indicate levels is selected from the group consisting of oxygenated haemoglobin, non-oxygenated haemoglobin, total haemoglobin, and cytochrome.

4. Apparatus according to claim 1 comprising a means for identifying a first time corresponding to an onset of voiding.

5. Apparatus according to claim 4 wherein the means for identifying the first time comprises a user interface control that can be manually operated to indicate an onset of voiding.

6. Apparatus according to claim 4 wherein the means for identifying the first time comprises means for detecting an onset signature in the sensor signals, the onset signature characteristic of the onset of voiding.

7. Apparatus according to claim 6 wherein the onset signature comprises a departure of a level indicated by one or more of the sensor signals from a baseline value.

8. Apparatus according to claim 4 wherein the means for identifying the first time comprises an input for receiving a signal from a bladder contraction detector.

9. Apparatus according to claim 8 comprising a bladder contraction detector connected to provide a signal indicative of bladder contractions to the input.

10. Apparatus according to claim 9 wherein the bladder contraction detector is configured to detect bladder contractions by directing energy towards the subject's bladder and detecting energy returning from the subject's bladder.

11-13. (canceled)

14. Apparatus according to claim 4 wherein the means for identifying the first time comprises a uroflow detector.

15. Apparatus according to claim 4 wherein the means for identifying the first time comprises a urine detector.

16. Apparatus according to claim 15 wherein the urine detector is configured to detect urine by detecting a change in electrical conductivity between two or more electrodes in a path of the urine.

17. Apparatus according to claim 4 wherein the means for identifying the first time comprises an EMG input for receiving an EMG signal and means for detecting in an EMG signal received at the EMG input an EMG signature that is characteristic of the onset of voiding.

18. Apparatus according to claim 4 comprising means for determining a second time corresponding to an end of voiding.

19. Apparatus according to claim 18 wherein the means for determining the second time comprises a user interface control that can be manually operated to indicate an end of voiding.

20. Apparatus according to claim 18 wherein the means for determining the second time comprises a timer configured to measure a predetermined time after an onset of voiding.

21. Apparatus according to claim 18 wherein the means for determining the second time comprises a flow detector and the analysis unit is configured to monitor an output of the flow detector to determine when uroflow has fallen to below a threshold value.

22. Apparatus according to claim 18 wherein the means for determining the second time comprises a urine detector and the analysis unit is configured to monitor an output of the urine detector.

23. Apparatus according to claim 18 wherein the means for determining the second time comprises means for detecting an end signature in the sensor signals, the end signature characteristic of the end of voiding.

24. Apparatus according to claim 1 comprising a sensor operable to generate the sensor signals wherein the sensor is selected from a trans-urethral sensor, a trans-rectal sensor and a trans-vaginal sensor.

25-29. (canceled)

30. Apparatus according to claim 1 wherein the analysis unit is configured to fit a function comprising one or more line segments to a curve indicating a variation of one of the levels with time.

31. Apparatus according to claim 30 wherein the function comprises from one to four connected line segments.

32. Apparatus according to claim 31 wherein the analysis unit is configured to fit one of a plurality of fitting functions to the curve, the plurality of fitting functions each comprising a different number of line segments.

33. Apparatus according to claim 32 when the plurality of fitting functions includes a first fitting function comprising one line segment, a second fitting function comprising two connected line segments, and a third fitting function comprising three connected line segments.

34. Apparatus according to claim 31 wherein the analysis unit is configured to select one of the plurality of fitting functions that provides a best fit to the curve.

35. Apparatus according to claim 30 wherein the analysis unit is configured to determine whether a slope of each line segment of the fitted function is positive or negative and to identify a fitted pattern in the slopes of the segments.

36. Apparatus according to claim 35 wherein the fitted pattern comprises a pattern selected from the group consisting of:
one line segment, negative slope;
one line segment, positive slope;
two line segments, positive slope followed by negative slope;
two line segments, positive slope followed by negative slope;
three line segments, positive slope followed by negative slope followed by positive slope;
three line segments, negative slope followed by positive slope followed by negative slope;
four line segments, positive slope followed by negative slope followed by positive slope followed by negative slope; and,
four line segments, negative slope followed by positive slope followed by negative slope followed by positive slope.

37. Apparatus according to claim 35 comprising a display wherein the apparatus is configured to display a visual representation of the fitted pattern.

38. Apparatus according to claim 30 wherein the analysis unit is configured to generate a display comprising the fitted function superposed on the curve.

39. Apparatus according to any claim 1 comprising a display wherein the apparatus is configured to display on the display one or more curves, each of the curves illustrating a time variation in the levels of a corresponding one of the one or more compounds.

40. Apparatus according to claim 39 configured to display on the display an onset indicia indicative of a time corresponding to an onset of voiding.

41. Apparatus according to claim 40 configured to display on the display an end indicia indicative of a time corresponding to an end of voiding.

42. Apparatus according to claim 18 wherein the analysis unit is configured to generate a possible diagnosis based upon the identified pattern and one or more of:
a residual amount of urine after urination;
a maximum uroflow rate; and,
a delay in onset of urination.

43. Apparatus according to claim 42 wherein the analysis unit comprises a logic system configured to select one of a predetermined plurality of possible diagnoses based at least in part on the identified pattern.

44. Apparatus according to claim 43 wherein the logic system is configured to identify one of a plurality of groups of the predetermined plurality of possible diagnoses based on the identified pattern and to select one of the predetermined plurality of possible diagnoses within the selected group based at least in part on one or more of a residual amount of urine after urination; a maximum uroflow; and, a delay in onset of urination.

45. Apparatus according to claim 44 wherein the logic system is configured to compare the maximum uroflow to a threshold and to select the one of the predetermined plurality of possible diagnoses within the selected group based at least in part on the comparison of the maximum uroflow to the threshold.

46. Apparatus according to claim 44 wherein the logic system is configured to compare the measured amount of residual urine to a threshold and to select the one of the predetermined plurality of possible diagnoses within the selected group based at least in part on the comparison of the amount of residual urine to the threshold.

47. Apparatus according to claim 43 wherein the predetermined plurality of possible diagnoses include diagnoses corresponding to: “normal”; “unobstructed”; “equivocal”; and “obstructed”.

48. Apparatus according to claim 1 comprising a data logger wherein the apparatus is configured to accumulate records of the levels of the one or more compounds for a plurality of voiding cycles for a subject and to generate the indicator of urinary tract function based on the records for the plurality of voiding cycles.

49. Apparatus according to claim 1 wherein the analysis unit is configured to identify a pattern in the time variations for a plurality of compounds and to generate the indicator of urinary tract function based at least in part on the plurality of identified patterns.

50. A method for monitoring urinary tract function in a subject, the method comprising:
detecting levels of one or more compounds in tissues of a bladder of the subject;
identifying a pattern in time variations of the detected levels; and
generating a possible diagnosis of urinary tract function based at least in part on the identified pattern.

51. A method according to claim 50, wherein detecting levels comprises detecting levels of one or more compounds selected from the group consisting of oxygenated haemoglobin, non-oxygenated haemoglobin, total haemoglobin, and cytochrome.

52. A method according to claim 50 comprising identifying a first time corresponding to an onset of voiding.

53. A method according to claim 52, wherein identifying the pattern comprises identifying time trends in the detected levels after the first time.

54. A method according to claim 53, wherein identifying the pattern comprises identifying an initial time trend in the detected levels after the first time and determining whether the initial time trend is upward or downward.

55. A method according to claim 53 wherein detecting levels comprises receiving sensor signals indicative of the levels of the one or more compounds.

56. A method according to claim 52 wherein identifying the first time comprises operating a manual input to indicate the onset of voiding.

57. A method according to claim 55, wherein identifying the first time comprises detecting an onset signature in the sensor signals, the onset signature characteristic of the onset of voiding.

58. A method according to claim 57, wherein detecting the onset signature comprises identifying a departure in the levels from a baseline value.

59. A method according to claim 52 wherein identifying the first time comprises receiving a signal from a bladder contraction detector.

60. A method according to claim 52 wherein identifying the first time comprises detecting uroflow.

61. A method according to claim 52 wherein identifying the first time comprises detecting a presence of urine.

62. A method according to claim 52 wherein identifying the first time comprises receiving an EMG signal and identifying an EMG signature characteristic of the onset of voiding.

63. A method according to claim 52 comprising identifying a second time corresponding to an end of voiding.

64. A method according to claim 63, wherein identifying the second time comprises operating a manual input to indicate the end of voiding.

65. A method according to claim 63, wherein identifying the second time comprises identifying when a predetermined time has lapsed after the onset of voiding.

66. A method according to claim 63, wherein identifying the second time comprises monitoring uroflow and determining when uroflow has fallen below a threshold value.

67. A method according to claim 63, wherein identifying the second time comprises monitoring a presence of urine and identifying a time when urine is no longer detected.

68. A method according to claim 55, comprising identifying a second time corresponding to an end of voiding, wherein identifying the second time comprises detecting an end signature in the sensor signals, the end signature characteristic of the end of voiding.

69. A method according to claim 50 wherein identifying the pattern comprises fitting a fitted function comprising one or more line segments to a curve indicating a time variation of one of the levels.

70. A method according to claim 69, wherein the fitted function comprises one to four connected line segments.

71. A method according to claim 70, wherein identifying the pattern comprises fitting a plurality of fitting functions to the curve, the plurality of fitting functions each comprising a different number of line segments.

72. A method according to claim 71, wherein fitting a plurality of fitting functions comprises fitting a first fitting function comprising one line segment, fitting a second fitting function comprising two connected line segments, and fitting a third fitting function comprising three connected line segments.

73. A method according to claim 71 comprising selecting one of the plurality of fitting functions that provides a best fit to the curve as the best fitted function.

74. A method according to claim 73, comprising determining whether a slope of each line segment of the best fitted function is positive or negative and identifying a fitted pattern in the slopes of the segments.

75. A method according to claim 74, wherein the fitted pattern comprises a pattern selected from the group consisting of:
one line segment, negative slope;
one line segment, positive slope;
two line segments, positive slope followed by negative slope;
two line segments, positive slope followed by negative slope;
three line segments, positive slope followed by negative slope followed by positive slope;
three line segments, negative slope followed by positive slope followed by negative slope;
four line segments, positive slope followed by negative slope followed by positive slope followed by negative slope; and,
four line segments, negative slope followed by positive slope followed by negative slope followed by positive slope.

76. A method according to claim 74 comprising displaying a visual representation of the fitted pattern on a display.

77. A method according to claim 76, comprising displaying the visual representation of the fitted pattern superposed on the curve.

78. A method according to claim 50 comprising displaying one or more curves on a display, each of the curves illustrating a time variation in the levels of a corresponding one of the one or more compounds.

79. A method according to claim 50 wherein generating the possible diagnosis is based upon the identified pattern and one or more of:
a residual amount of urine after voiding;
a maximum uroflow rate during voiding; and,
a delay in the onset of voiding.

80. A method according to claim 50 comprising generating a plurality of possible diagnoses based at least in part on the identified pattern, the plurality of possible diagnoses selected from a plurality of predetermined possible diagnoses.

81. A method according to claim 80, comprising selecting one of the predetermined plurality of possible diagnoses based at least in part on one or more of:
a residual amount of urine after voiding;
a maximum uroflow rate during voiding; and,
a delay in the onset of voiding.

82. A method according to claim 81, comprising:
determining maximum uroflow rate during voiding;
comparing the maximum uroflow rate to a threshold value; and
selecting one of the predetermined plurality of possible diagnoses based at least in part on the comparison of the maximum uroflow rate to the threshold value.

83. A method according to claim 81, comprising:
determining a residual amount of urine after voiding;
comparing the residual amount to a threshold value; and
selecting one of the predetermined plurality of possible diagnoses based at least in part on the comparison of the residual amount to the threshold value.

84. A method according to claim 80 wherein the plurality of predetermined possible diagnoses include diagnoses corresponding to “normal”, “unobstructed”, “equivocal” and “obstructed”.

85. A method according to claim 50 wherein detecting levels of one or more compounds comprises receiving sensor signals indicative of the levels of the one or more compounds from a sensor selected from the group consisting of:
a trans-urethral sensor;
a trans-vaginal sensor; and
a trans-rectal sensor.

86. (canceled)

87. (canceled)
